(19) United States
(12) Patent Application Publication
(10) Pub. No.: US 2010/0056833 A1
(43) Pub. Date: Mar. 4, 2010

(54) PRETREATMENT OF BIOLOGICAL FEEDSTOCKS FOR HYDROCONVERSION IN FIXED-BED REACTORS

(76) Inventors: Julio Suarez, Broken Arrow, OK (US); Ramin Abhari, Bixby, OK (US); Brad Bunch, Tulsa, OK (US)

Correspondence Address: Hall, Estill, Hardwick, Gable, Golden &;Nelson, P.C., 100 North Broadway, Chase Tower, Suite 2900, Oklahoma City, OK, 73102, US

(21) Appl. No.: 12/201,109

(22) Filed: Aug. 29, 2008

(51) Int. Cl. C07C 1/00

(52) U.S. Cl. 585/240

(57) ABSTRACT

A process for pretreating biological feedstocks for hydroconversion in a fixed-bed reactor. A feed stream having free fatty acids, fatty acid esters, or combinations thereof is contacted with a citric acid solution. The biological feedstock is separated from the aqueous solution to efficiently produce a pretreated biological feedstock substantially absent of metals and phosphorus.

### STATEMENT REGARDING FEDERALLY SPONSORED RESEARCH OR DEVELOPMENT

Not applicable.

### CROSS-REFERENCE TO RELATED APPLICATIONS

Not applicable.

### BACKGROUND OF THE INVENTION

The present invention relates to pretreatment of biological feedstocks for conversion to hydrocarbons. More specifically, the present invention relates to removal of solubilized metals and phosphorus from fatty acid and/or glycerides.

Biomass is a renewable alternative to fossil raw materials in production of liquid fuels and chemicals. Development of more efficient biomass conversion processes is considered a key step toward wider use of renewable fuels.

Vegetable oils, animal fats, and bio-derived greases make particularly attractive renewable feedstocks. From a physical property stand point, these feeds are liquids or low melt point solids, and therefore easily transported through pipe networks. Chemically, these have a relatively high carbon content and hence relatively high energy content. However, these feeds also contain metals, chlorides, and phosphorus that can be deleterious to the performance of hydroconversion catalysts. Chlorides and phosphorus can alter the activity of the catalyst. Solubilized metals, such as calcium and sodium, can deposit on the catalyst as the solubility character changes with transition from acids/esters to hydrocarbons in a reactor. In particular, this can lead to rapid plugging of fixed-bed reactors.

Several prior art processes for producing hydrocarbons from starting materials such as plants and animals are known. U.S. Pat. No. 2,163,563 issued to Schrauth teaches a hydrogenation process for converting animal fats into hydrocarbons of mineral oil character.

U.S. Pat. No. 4,992,605 issued to Craig and Soveran discloses a hydrogenation process for converting vegetable oils to mainly C15-C18 n-paraffins.

U.S. Patent Application 2006/0207166 filed by Herkowitz, et al. teaches hydroconversion of animal fats and vegetable oils into a diesel fuel composition including linear and branched paraffins, alkyl benzene, and alkyl cyclohexane. None of the preceding art teaches pretreatment of the feed.

U.S. Patent Application 2006/0264684 filed by Petri and Marker teaches that alkali metals, phosphorus, and other contaminants in the biological feedstock may be partially removed before hydrogenation. Two means of removing the alkali metals and phosphorus are mentioned therein: (1) treatment with ion-exchange resins and (2) contacting with an acid such as sulfuric, nitric or hydrochloric acid in a reactor. However the single-stage removal efficiencies for individual metals is in the 32% to 75% range. Furthermore, the remaining metals and phosphorus in the pretreated feed was 310 wppm which is considered unacceptably high for efficient hydroconversion in a fixed-bed reactor. For achieving commercially viable fixed-bed reactor run lengths, single-stage removal efficiencies above 90%, and residual total metals and phosphorus content below 100 wppm are desired.

To this end, although processes of the existing art utilize biomass to produce hydrocarbons, and the importance of feed pretreatment is recognized therein, further improvements are desirable to provide a new process for preparing biological feedstocks to make fuels and chemicals.

### BRIEF DESCRIPTION OF THE DRAWINGS

FIG. 1 is a schematic diagram of a feed pretreatment process according to the present invention.

FIG. 2 is a graphical representation of the correlation between ash and metals analysis.

### SUMMARY OF THE INVENTION

Vegetable oils, animal fats, and bio-derived greases are glycerides (mainly tri- and di-glycerides) with varying concentrations of free fatty acids. Tall oil from pine tree is concentrated in fatty acids known as tall oil fatty acids.

The conversion of vegetable oils, animal fats, tall oil fatty acids, tall oil, and/or greases (also known as “biological feedstocks”) to fuels and chemicals may be achieved via hydroprocessing. Generically referred to as hydroconversion, the hydroprocessing reactions include hydrogenation, hydrodeoxygenation, hydrodesulfurization, hydrodenitrification, hydroisomerization, and hydrocracking.

Solubilized metals and phosphorus in a biological feedstock reduce the performance of hydroconversion reactions. In particular, in the commonly used fixed-bed hydroconversion reactors, the solubilized metals deposit in a void space of the reactor bed. The deposit of solubilized metals leads to a rapid increase in pressure drop across the reactor bed and short run lengths making hydroconversion of these feeds commercially unviable.

In the inventive process disclosed herein, the solubilized metals and phosphorus are effectively removed from the biological feedstocks. The biological feedstocks are washed with dilute citric acid solution to produce a pretreated biological feedstock substantially free of metals and phosphorus and well suited for efficient hydroconversion.

### DETAILED DESCRIPTION OF THE INVENTION

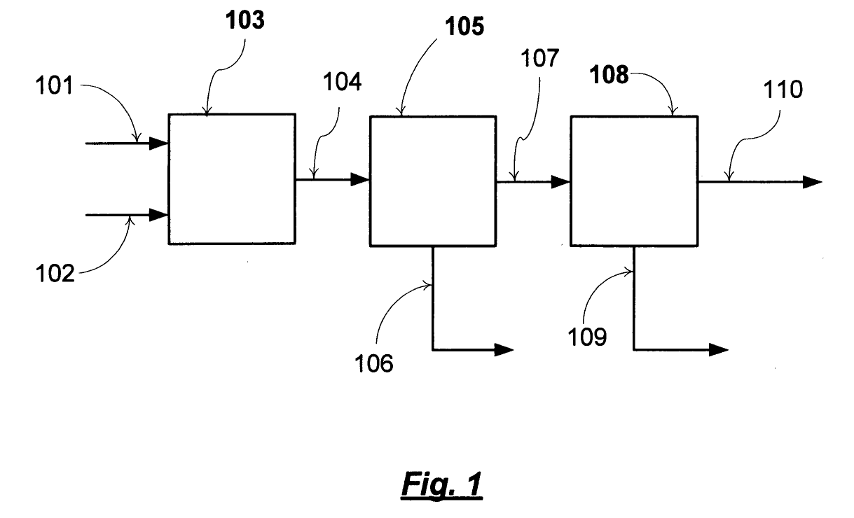

Referring now to the drawings, and more particularly to FIG. 1, shown therein is a schematic of one embodiment of the operation of the process in accordance with the present invention as described herein. A biological feedstock 101 and a citric acid solution 102 are fed into a contacting device 103. The biological feedstock 101 is optionally pre-filtered before entering the contacting device 103.

The concentration of the citric acid solution 102 is from about 0.5 wt. % to about 20 wt. %, preferably from about 5.0 wt. % to about 15 wt. % (mass citric acid per mass aqueous solution). The volumetric ratio of biological feedstock to citric acid solution is from about 20:1 to about 2:1, preferably from about 5:1 to about 15:1. It should be understood by one of ordinary skill in the art that although a citric acid solution is disclosed as being utilized in the present process, any acid, such as phosphoric acid, sulfuric acid, hydrochloric acid, nitric acid, acetic acid or carbonic acid, may be used so long as the acid functions in accordance with the present invention as described herein. During pre-filtration and acid contacting, the temperature of the biological feedstock is maintained from about 140° F. to about 280° F.

The contacting device 103 functions to contact the biological feedstock 101 with the citric acid solution 102. It should be understood by one of ordinary skill in the art, that any device capable of producing intimate contacting between two immiscible phases may be used in the present invention. Specific examples of contacting devices suitable for this application include mixing valve, static mixer, or roto-stator high shear mixer.

Stream 104, the two phase effluent from the contacting device 103, is fed to a liquid-liquid separator 105. The geometry and size of the separator 105 allow for most of the small aqueous droplets formed in the contracting device 103 to coalesce and form larger droplets that separate from a washed biological feedstock stream 107. The separator is sized to provide between about 5 to about 30 minutes hold up time for the fluid. Preferred operating conditions for the separator are between about 140° F. and about 280° F. at pressures less than about 500 psig. An aqueous stream 106 from the separator 105 contains citric acid, metal cations, chloride anions, soluble citric acid metal complexes, and small amounts of insoluble complexes.

The washed biological feedstock stream 107 contains very small water droplets and is fed into and processed through a coalescing filter 108 to achieve further separation of spent aqueous citric acid complexes. The coalescing filter operates at temperatures between about 140° F. and about 280° F. at pressures less than about 500 psig. The aqueous stream 109 is compositionally the same as stream 106. Although the filter 108 is utilized to further separate the spent aqueous citric acid complexes, it should be understood by one of ordinary skill in the art that the separation of the very small water droplets in the washed biological feedstock 107 may also be achieved by a centrifuge, an electrical grid or any other means known in the art used to separate liquids.

Spent aqueous streams 106 and 109 are optionally combined and sent to a citric acid reclamation unit (not shown) and/or partially recycled. The washed and water-separated biological feedstock stream 110 has a total metals and phosphorus content less than about 100 ppm, preferably less than about 50 ppm, and more preferably less than about 20 ppm. The metals include calcium, iron, potassium, magnesium, and sodium. The pretreated biological feedstock stream 110 is optionally transported to a surge drum (not shown) for pumping to a hydroconversion reactor system (not shown) which optionally includes a fixed-bed reactor for converting pretreated biological feedstock to hydrocarbons.

Although the pretreatment process as illustrated in FIG. 1 is shown utilized for a single-stage continuous operation, it should be understood by one of ordinary skill in the art that the required mixing and liquid-liquid phase separation may also be conducted in batch cycles. One of ordinary skill in the art will further recognize that the continuous operation may employ a plurality of contactor-separator stages, with counter-current, cross-current, or co-current flow of the citric acid solution 102.

In order to further illustrate the present invention, the following examples are given. However, it is to be understood that the examples are for illustrative purposes only and are not to be construed as limiting the scope of the subject invention.

### EXAMPLES

### Example 1

### Measuring Metal Contaminants by Ash Analysis

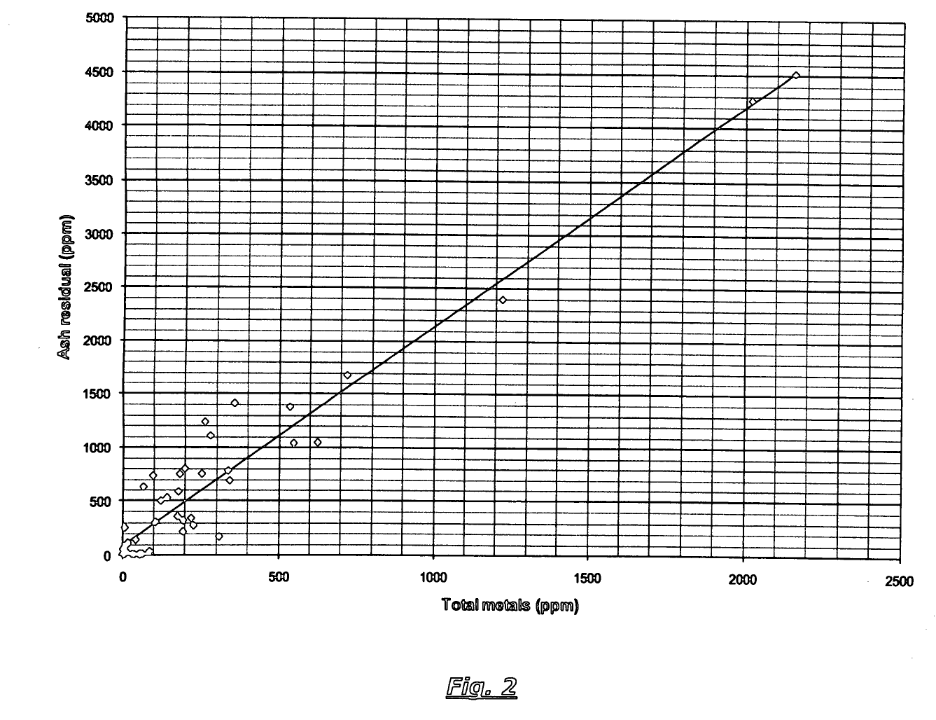

Upon ignition, the solubilized metals contained in biological feedstocks remain as ash. Although ash may also contain other non-combustible inorganic matter, it is proportional to the level of metal contaminants in the biological feedstock. The process of measuring ash consists of (1) weighing about 100 g of homogenized biological feedstock by an analytical balance, (2) placing the biological feedstock in a tared crucible, (3) melting the contents of the crucible over a hot plate, (4) igniting the molten biological feedstock in the crucible in a hood using proper personal protection and associated safe practices, and (5) weighing the crucible. Net weight of ash remaining in the crucible divided by weight of the biological feedstock placed therein gives the ash content. The ash analysis was conducted on biological feedstocks of various metals content. FIG. 2 shows a graphical representation of the correlation between ash and total Group I and Group II metals as analyzed by Inductively Coupled Plasma (ICP) Atomic Emission Spectroscopy.

### Example 2

### Hydrotreater Operation with a Relatively High-Contaminant Biological Feedstock

Four 100 cc tubular reactors were each loaded with 80 cc of a commercial NiMo catalyst and 20 cc of 70-100 mesh glass beads. The NiMo catalyst was sulfided with a dimethyl disulfide (DMDS) solution under H₂ flow conditions. Decomposition of DMDS to hydrogen sulfide was confirmed by use for lead acetate before the reactor temperature was raised from the first hold temperature of about 400° F. to the final hold temperature of about 650° F. The total sulfiding cycle (start to end of DMDS solution flow) was about 20 hrs.

After a 48 hr catalyst break-in, a triglyceride feed with relatively high solublized metal contaminants, inedible tallow, was introduced to the reactor. The properties of the feedstock, including contaminant metals and ash (non combustible inorganics), are summarized in Table 1. The operating conditions for all four reactors were: 1 liquid hourly space velocity (LHSV), 10,000 SCF/bbl gas-to-oil ratio (GOR), 700° F., and 1,200 psig. The waxy solid tallow feed was thus converted to a clear liquid. Full conversion of tallow to hydrocarbons was confirmed with gas chromatography (GC).

About twenty-four hours after start of the tallow feed, two of the four reactors experienced high pressure drop. This ultimately led to a drop in gas flow rate and conversion performance.

TABLE 1
Level of ash (non-combustible inorganic contaminants) in
feedstocks of Examples 2 and 3

| | Example 2 | Example 3 |
|---|---|---|
| Feedstock | Inedible Tallow | Partially Hydrogenated |
| | | Soybean Oil |
| Appearance | Light tan solid | White semi-solid |
| Ash (wppm) | 749 | 19 |
| Days before spike in | 2 | 50+ |
| reactor delta-P | | |

### Example 3

### Hydrotreater Operation with a Low-Contaminant Biological Feedstock

The reactors of Example 2 were reloaded with catalyst and sulfided using the same procedure as discussed in Example 2. One of the reactors contained the same grade of catalyst used in Example 2. After a catalyst break-in, a triglyceride feedstock with low contaminant content, partially hydrogenated soybean oil, was introduced to the reactor. As indicated by the ash values of Table 1, this feedstock had 97.5% less contaminant than the triglyceride feedstock of Example 2.

The hydrotreater reactors were operated under the same liquid and gas flow conditions as Example 2. Complete conversion of feedstock to hydrocarbon product was confirmed by GC at all operating temperatures tested: 550° F., 600° F., 650° F., 700° F., and 750° F. The reactor system was operated for 50 days on the same feed without increase in pressure drop across any of the four reactors.

### Example 4

### Washing a Fat/Grease Blend with Water

A biological feedstock was prepared by blending waste fats and greases according to Table 2.

TABLE 2
Make up of biological feedstock of Examples 4 and 5

| Fat/Grease Type | Mass Percent |
|---|---|
| Poultry Fat | 46% |
| Yellow Grease | 18% |
| Brown Grease | 18% |
| Floatation Grease | 9% |
| Misc. Animal Fat | 9% |

The contaminants present in this biological feedstock are summarized in Table 3.

TABLE 3
Contaminants present in the biological feedstock
of Examples 4 and 5

| Feedstock attribute/- | Concentration |
|---|---|
| contaminant | (wppm) |
| Ash | 1,675 |
| Calcium | 285 |
| Iron | 67.3 |
| Potassium | 117 |
| Magnesium | 7.6 |
| Sodium | 123 |
| Phosphorus | 144 |
| Silicon | 3.2 |
| Zinc | 3.6 |
| Acid number (mg KOH/g) | 94.7 |

The fat/grease feed blend was filtered through a 10 mm bag filter. The ash value of the filtered product was measured as 1,715 wppm—essentially unchanged. The filtered feedstock was then washed with de-mineralized water in a continuous operation. The biological feedstock to water volumetric flow ratio was 10:1. The two streams, fat/grease at 15 gal/min and water at 1.5 gal/min, were brought into contact in a mix tee. The washed fat/grease blend was measured for ash content, and the wash cycle was repeated under the same conditions. The results of each water wash cycle are summarized in Table 4. Based on the ash analyses, even after two water wash cycles the inorganics/metals content remained mostly unchanged.

TABLE 4
Clean-up performance with water wash cycles

| Feedstock attribute/- | | |
|---|---|---|
| contaminant | Water Wash 1 | Water Wash 2 |
| Ash (wppm) | 1,253 | 1,090 |
| Ash Component Removal per Cycle | 25.2% | 12.8% |
| Acid Value (mg KOH/g) | 118 | 121 |
| Moisture and Volatiles (wt %) | 4.2 | 2.0 |

### Example 5

### Washing a Fat/Grease Blend with Citric Acid

After the second water wash cycle, the fat/grease blend as shown in Table 3 was washed with 10% citric acid solution. The same continuous washing operation described in Example 4 was used, with a 10:1 ratio of fat/grease to aqueous citric acid solution. The properties of the filtered citric-washed product are summarized in Table 5. It is evident from the ash analyses that citric acid wash removed most of the inorganic/metal contaminants.

TABLE 5
Clean-up performance with citric acid wash

| Feedstock attribute/- | | |
|---|---|---|
| contaminant | Before | After |
| Ash (wppm) | 1,090 | 67.2 |
| Ash Component Removal per Cycle | — | 93.8% |
| Acid Value (mg KOH/g) | 121 | 129 |
| Moisture and Volatiles (wt %) | 2.0 | 1.3 |

From the above description, it is clear that the present invention is well adapted to carry out the objects and to attain the advantages mentioned herein as well as those inherent in the invention. While presently preferred embodiments of the invention have been described for purposes of this disclosure, it will be understood that numerous changes may be made which will readily suggest themselves to those skilled in the art and which are accomplished within the spirit of the invention disclosed and claimed.

What is claimed is:

1. A process for pre-treating biological feedstocks for hydroconversion comprising the steps of:
(a) providing a biological feedstock stream containing free fatty acids, fatty acid esters, or combinations thereof;
(b) contacting the biological feedstock stream with an aqueous acid solution; and
(c) separating an aqueous phase to yield a pre-treated biological feedstock having an ash content less than about 100 wppm.

2. The process of claim 1 wherein the biological feedstock comprises animal fat and yellow grease.

3. The process of claim 1 further comprising the step of:
filtering the biological feed stock stream.

4. The process of claim 1 wherein the acid is citric acid.

5. The process of claim 4 wherein the concentration of the citric acid solution is at least 0.5 wt. %.

6. The process of claim 1 wherein the acid is sulfuric acid, phosphoric acid, hydrochloric acid, nitric acid, acetic acid, or carbonic acid.

7. The process of claim 1 further comprising the step of:
feeding the pre-treated biological feedstock to a hydroconversion unit.

8. The process of claim 7 wherein the hydroconversion unit includes a fixed-bed reactor.

9. The process of claim 8 wherein the fixed-bed reactor converts pre-treated biological feedstock to hydrocarbons.

10. The process of claim 1 wherein the ash content of the biological feedstock is reduced by at least 90%.

11. The process of claim 1 wherein the pretreated biological feedstock having an ash content less than about 80 wppm ash.

12. The process of claim 1 wherein the pre-treated biological feedstock having an ash content less than about 50 wppm ash.

13. The process of claim 1 wherein the total metals and phosphorus of the pretreated biological feedstock is less than about 40 wppm

14. The process of claim 13 wherein the metals include calcium, iron, potassium, magnesium, and sodium.

15. The process of claim 1 further comprising the steps of:
providing a plurality of contacting steps; and
providing a plurality of separating steps.
